(19) United States
(12) Patent Application Publication
(10) Pub. No.: US 2003/0105519 A1
(43) Pub. Date: Jun. 5, 2003

(54) ARTIFICIAL CHORDAE REPLACEMENT

(76) Inventors: ROLAND FASOL, WOLLBACH (DE); MARVIN J. SLEPIAN, TUCSON, AZ (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 09/148,819

(22) Filed: Sep. 4, 1998

Related U.S. Application Data
(63) Continuation-in-part of application No. 08/923,892, filed on Sep. 4, 1997, now abandoned.

(51) Int. Cl. A61F 2/24

(52) U.S. Cl. 623/2.1; 623/13.11; 606/228

(57) ABSTRACT

Artificial chordae having a strand member and a first and second pair of sutures at either longitudinal end of the strand member. The artificial chordae is preferably a unitary unit, formed from inelastic flexible material. In one embodiment, the artificial chordae comprises multiple strand members joined together at a joined end. Different sized artificial chordae are provided sized to fit the patient's heart. The appropriately sized artificial chordae is chosen by using a chordae sizing gauge having a shaft and a transverse member, to measure the space within the patient's heart where the artificial chordae is attached. The artificial chordae

This application is a continuation-in-part application of prior co-pending application U.S. Ser. No. 08/923,892, filed Sep. 4, 1997, entitled Artificial Chordae Replacement.

### BACKGROUND OF THE INVENTION

This invention relates to an artificial chordae device, and more particularly to an artificial chordae replacement for a mitral or tricuspid valve.

A vertebrate heart consists of four cavities, known as the left and right atria and the left and right ventricles. Oxygenated blood from the lungs is received by the left atrium, and passes into the left ventricle which forces it via the aorta to the tissues of the body. Blood returning from the body tissues is received by the right atrium, and passes into the right ventricle which forces it to the lungs to be oxygenated. A valve, known as the mitral or bicuspid valve, regulates the flow of blood between the left atrium and ventricle, whereas the tricuspid valve serves the same function for the right atrium and ventricle. The mitral valve is a thin continuous membrane having two indentations dividing it into two principal trapezoidal leaflets of unequal size. Tendinous strands known as chordae tendineae connect the edges of the valve leaflets to the papillary muscle on the ventricular surface, so that relaxation and contraction of the left ventricle will act on the mitral valve causing it to open and close. Furthermore, the subvalvular structures, e.g. the papillary muscles and chordae tendineae, play an important role in structuring the geometry of the heart and ventricular function.

Heart valve replacement is a well known procedure in which an artificial heart valve prostheses is implanted in place of a diseased or malfunctioning heart valve. While artificial mechanical, man made, valves are generally durable, the patient may be prone to infection and must be treated with anticoagulant medications for the rest of their lives to prevent thromboembolic complications or thrombotic occlusion of the prosthesis. Moreover, anticoagulation therapy may cause life threatening complications, and is responsible for a high percentage of lethal and nonlethal heart valve complications. The need for anticoagulation therapy can be avoided in general by the use of artificial biological heart valves, such as bovine xenografts. Nevertheless, dystrophic calcification with subsequent degeneration is the major cause of failure of such bioprostheses in the long term, and bioprosthetic valve dysfunction may cause precipitous clinical deterioration requiring reoperation in a high percentage of patients. Additionally, when mitral or tricuspid valve replacement is performed, the chordae are cut, thus leaving the geometry and function of the ventricle impaired and in need of reconstruction.

As an alternative to conventional heart valve replacement operations, a high percentage of patients could be treated with repair including the repair of diseased and malfunctioning heart valve tendineae chordae. Such reconstructive heart valve operations generally don't require anticoagulation therapy, and the patient's can expect a significantly reduced risk of postoperative complication with a subsequently higher life expectancy. However, heart valve tendineae chordae repair operations are technically demanding. In general, the present way of replacing a chordae uses a simple suture with one needle on each end of the suture. The suture is stitched through the papillary muscle and secured thereto with a knot. The two ends of the suture are then similarly stitched through the free ends of the valve leaflets. However, in attempting to tie a second knot to secure the suture to the valve leaflets, because there is nothing holding the suture in place, the length of the suture spanning the distance between the papillary muscle and valve leaflet is likely to change. This complication increases the skill and time required to perform the procedure. Moreover, the valve will not function properly if the length of the artificial chordae between the papillary muscle and valve leaflet is overly long or overly short.

Therefore, what has been needed is an artificial chordae replacement for the mitral and tricuspid valves which is easily secured in place between the papillary muscle and valve leaflet, and which will not allow for a change of length during the attachment process. Additionally, a need exists for easy and secure reconstruction of the subvalvular structures during valve replacement. The present invention satisfies these and other needs.

### SUMMARY OF THE INVENTION

The invention is directed to an artificial heart valve chordae, a heart valve chordae sizing gauge, and a method of using both to replace chordae in a heart valve. The artificial chordae of the invention is suitable for use in both the mitral and tricuspid heart valves.

The artificial heart valve chordae of the invention generally comprises a strand member with two sutures on each end of the member. One pair of sutures is used to attach the first end of the strand to the papillary muscle while the other pair of sutures attaches the second end to the edge of the valve leaflets. In one embodiment, an artificial chordae having one end for attachment to the papillary muscle (or valve leaflet) and multiple ends for attachment to multiple locations on the valve leaflets (or papillary muscle) is provided by an artificial chordae comprising at least two strand members side by side, or longitudinally juxtaposed, and joined together at one end. At the end where the strands are joined together is one pair of sutures for attaching that end to the papillary muscle (or valve leaflet), and at the free end of each strand is a pair of sutures for attaching that free end to a separate location on the valve leaflet (or papillary muscle).

The artificial chordae are formed from inelastic flexible material that is bioincorporable, such as TEFLON® (expanded polytetrafluoroethylene), or other suitable materials. A presently preferred embodiment has the strand member and sutures formed as a unitary one piece unit, which minimizes the risk of a rupture forming in the artificial chordae during use.

Once the artificial chordae is sutured into place, the length of the strand member defines the length of the implanted artificial chordae. The artificial chordae of the invention come in a variety of preset sizes with strand members having different fixed lengths, so that an artificial chordae can be chosen which has a length that is approximately equal to the distance between the site of implantation of the papillary muscle and valve leaflet where the artificial chordae will be attached. This configuration, having a strand member that is a fixed length sized to fit the patient's heart with suture pairs at each end of the member, is a substantial advance. The configuration provides for easy attachment and prevents a disadvantageous change in the artificial chordae length during attachment.

Because the artificial chordae is sized to fit the patient's heart, the distance between the patient's papillary muscle and valve leaflet is measured in order to select the appropriately sized artificial chordae. One aspect of the invention provides a heart valve chordae sizing gauge used to measure the distance between the papillary muscle and valve leaflet where the artificial chordae will be attached. The sizing gauge generally comprises a shaft with a transverse member, or tab. By holding the sizing gauge between the papillary muscle and valve leaflet at the desired location of the artificial chordae, the distance between the transverse member and one end of the shaft is used to approximate the length of the artificial chordae which is required. The transverse member is fixed to the shaft, so the sizing gauge is provided in a variety of different sizes in which the distance between the transverse member and the ends of the shaft vary.

In making the measurement, the physician is likely to try more than one differently sized sizing gauge until a gauge is found in which the distance between the transverse member and one end of the shaft is approximately equal to the distance between the papillary muscle and valve leaflet edge. Moreover because the distance between the papillary muscle and valve leaflet edge is not uniform, the physician measures the maximum and minimum distance so that an artificial chordae is chosen having a length that is between that maximum and minimum distance. In an alternative embodiment, the transverse member is slidably mounted on the shaft, to allow for adjustment of the distance between the transverse member and the end of the shaft during measurement.

In the surgical operation, the distance between the papillary muscle and the edge of the valve leaflet is measured with the heart valve chordae sizing gauge of the invention. Then, an artificial chordae having the appropriate strand length is chosen and attached in place using the pairs of sutures. One pair of sutures is threaded through the papillary muscle and tied into a knot, while a similar procedure is performed at the valve leaflet with the pair of sutures on the opposite end of the strand member. An identical procedure is used for the artificial chordae embodiment of the invention having multiple strand members joined together, except that a separate pair of sutures must be attached to the heart tissue for the free end of each strand member.

An identical procedure is performed in the case of valve replacement, except that one pair of sutures is placed through the valve annulus of the heart valve prosthesis before implanting the heart valve prosthesis, and then the second pair of sutures is attached to the papillary muscle.

The artificial chordae of the invention has superior ease of attachment due to the pair of sutures on each end of the strand member, so that the strand member defines the fixed length of the implanted artificial chordae. The invention thus avoids a change in the length of the artificial chordae during attachment, and therefore the risk of an improperly sized and possibly inoperative artificial chordae being attached. Furthermore, in the case of mitral or tricuspid valve replacement, the artificial chordae of the invention allows for easy and secure reconstruction of the subvalvular structures. These and other advantages of the invention will become more apparent from the following detailed description of the invention and the accompanying exemplary drawings.

### BRIEF DESCRIPTION OF THE DRAWINGS

FIG. 1 illustrates a conventional artificial chordae of the prior art.

FIG. 2 is an elevational view of an artificial chordae which embodies features of the invention.

FIG. 3 is an elevational view of one embodiment of an artificial chordae having multiple strand members.

FIG. 4 is an elevational view of a sizing gauge of the invention.

FIG. 5 illustrates a sizing gauge of the invention in use, positioned between a papillary muscle and a valve leaflet edge.

FIG. 6 is a schematic sectional view of a human heart.

FIG. 7 is an enlarged sectional view of the mitral valve of a human heart.

FIGS. 8a and 8b illustrate a sequence of steps in the attachment of the prior art artificial chordae.

FIGS. 9a and 9b illustrate a sequence of steps in the attachment of an artificial chordae of the invention.

FIG. 10 illustrates an artificial heart valve prosthesis.

FIG. 11 is an elevational view of an artificial chordae which embodies features of the invention having a pledget at one end of each pair of sutures.

FIG. 12 is an elevational view of one embodiment of an artificial chordae having multiple strand members and having a pledget at one end of each pair of sutures.

FIGS. 13a-13c illustrate one embodiment in which the strand member is folded.

FIG. 14 illustrates the folded strand member shown in FIG. 13c having a pin connecting the folds together.

FIG. 15 illustrates the folded strand member shown in FIG. 13c having a ring connecting the folds together.

FIG. 16 illustrates the folded strand member shown in FIG. 13c having a clip connecting the folds together.

FIG. 17 illustrates an artificial chordae assembly which embodies features of the invention being attached to a patient's mitral valve leaflet and papillary muscle, and having a stopping member comprising a clip on the second pair of sutures.

FIG. 18 illustrates an alternative embodiment of an artificial chordae assembly which embodies features of the invention, having a stopping member comprising a securable tube on the second pair of sutures.

FIG. 19 illustrates an alternative embodiment of an artificial chordae which embodies features of the invention having a suture and stopping members thereon and being attached to a patient's mitral valve leaflet and papillary muscle.

### DETAILED DESCRIPTION OF THE INVENTION

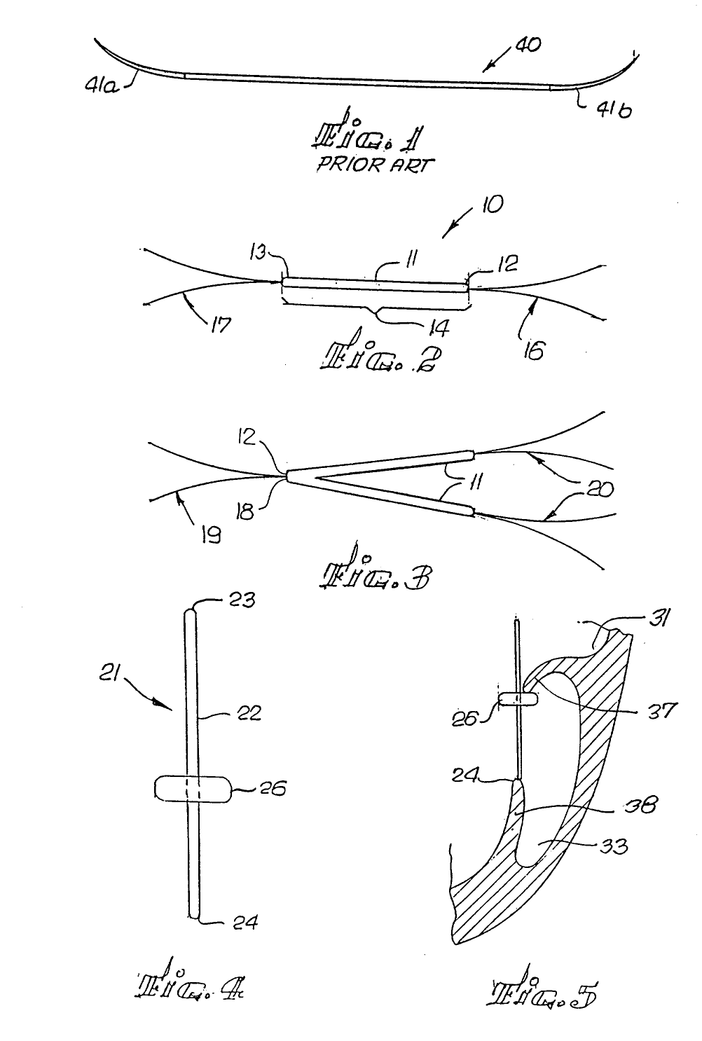

FIG. 1 illustrates a conventional chordae replacement suture 1 of the prior art, and needles 2a, b attached to the end of each suture.

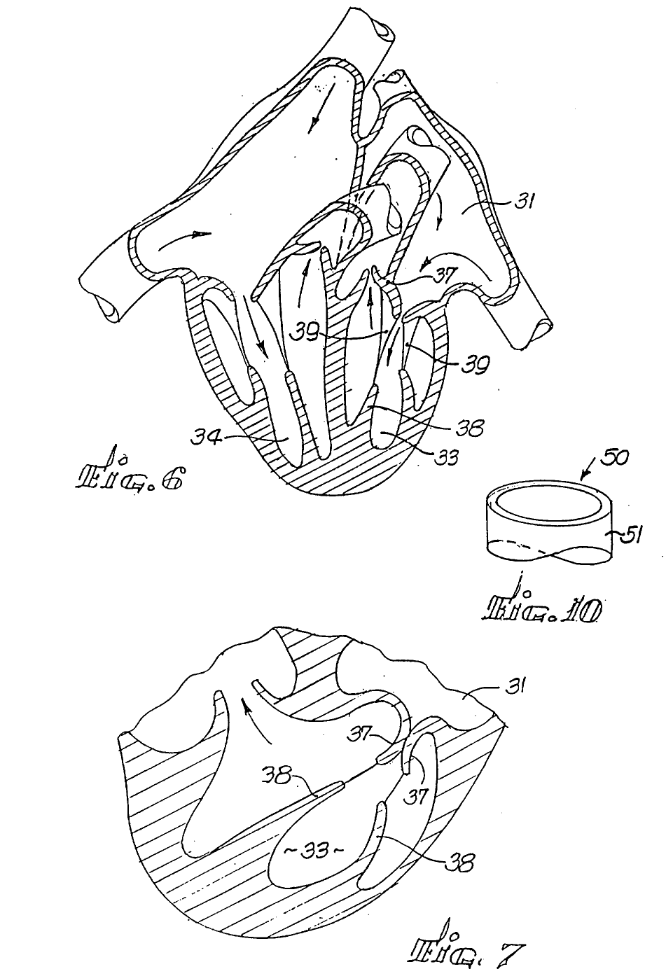

The artificial heart valve chordae 10 of the invention is illustrated in FIG. 2, and comprises at least one strand member 11 having a first end 12 and a second end 13, and a longitudinal portion 14. A first pair of sutures 16 extends from the strand member first end 12, and a second pair of sutures 17 extends from the strand member second end 13. One embodiment of the invention having multiple strand members 11 is illustrated in FIG. 3, and comprises at least two strand members 11 having a joined end 18. The strand member first ends 12 are fixed together to form the joined end 18, and the strand members 11 are longitudinally juxtaposed so that the strand longitudinal portions 14 are adjacent one another. One pair of sutures 19 extend from the joined end 18, and pairs of sutures 20 extend from the second end of each strand member. The strand members 11 joined together may have different longitudinal lengths, or may have substantially equal lengths.

For attaching the artificial chordae 10 to the patient's heart tissue, the end of each suture 16 would be provided with needles (not shown). The sutures 16, which may be from about 75 cm to about 90 cm in length, typically about 75 cm, may be surgically attached in the heart to attach the artificial chordae. The artificial chordae 10 is provided in different sizes having strand members 11 of various lengths. It is the size of the strand member 11 which defines the length of the implanted artificial chordae in place in the patient's heart. The strand member 11 is configured to extend from the papillary muscle to a location on the heart valve, and may be about 1 cm to about 6 cm in length, depending on the size of the heart as well as the point of placement chosen by the surgeon. The strand member 11 has a diameter of about 0.1 mm to about 0.25 mm, typically about 0.15 mm.

In a presently preferred embodiment, the strand member 11 and sutures 16, 17 of the artificial chordae are formed from a unitary unit. However, the strand and sutures may be formed as separate units joined together, and possibly from different materials. The artificial chordae is formed from biocompatible material that is relatively inelastic and flexible, to allow easy movement of the valve leaflets during opening and closing of the valve. The presently preferred material is TEFLON®, or expanded polytetrafluoroethylene, although it would be obvious to one skilled in the art that there are other suitable materials, including those which are frequently used to form sutures. The expanded polytetrafluoroethylene may be suture material or fabric material.

One aspect of the invention provides a heart valve chordae sizing gauge 21 for measuring the distance between the papillary muscle 38 and the valve leaflet edge 37. The sizing gauge 21 is illustrated in FIG. 4, and comprises a shaft 22 having a first end 23, a second end 24, and a transverse member 26 spaced a distance between the shaft first and second ends. The transverse member 26 is fixed to the shaft, and the sizing gauge 21 is provided in different sizes which correspond to the different sized artificial chordae 10. The size of the sizing gauge 21 is defined by the distance between the transverse member 26 and the shaft ends 23, 24. The sizing gauge 21 is formed from biocompatible material, and is preferably formed from a plastic material.

An alternative embodiment provides the transverse member 26 slidably mounted so as to slide along the shaft 22, so that the size of the sizing gauge 21 can be adjusted during the measurement. A means to releasably lock the slidable transverse member 26 onto the rod is provided. In the embodiment shown in FIG. 4, frictional engagement is used to lock the slidable transverse member onto the rod, although there are a variety of suitable locking mechanisms, including a compression fit clamp, screw clamp, and the like.

When the size of the artificial chordae is to be chosen, the physician measures the maximum and minimum distance between the papillary muscle 38 and valve leaflet edge 37, in order to choose an artificial chordae 10 with the correct size that is somewhere between the maximum and minimum lengths measured.

To make the measurements, the physician positions the sizing gauge 21 in place between the papillary muscle 38 and valve leaflet edge 37 (FIG. 5). The distance between the muscle 38 and leaflet edge 37 is then compared to the distance between the transverse member 26 and the shaft end, preferably the shaft second end 24. If necessary, the sizing gauge is exchanged for a sizing gauge of a different size until the distance between the muscle 38 and leaflet edge 37 is approximately equal to the distance between the transverse member 26 and the shaft second end 24.

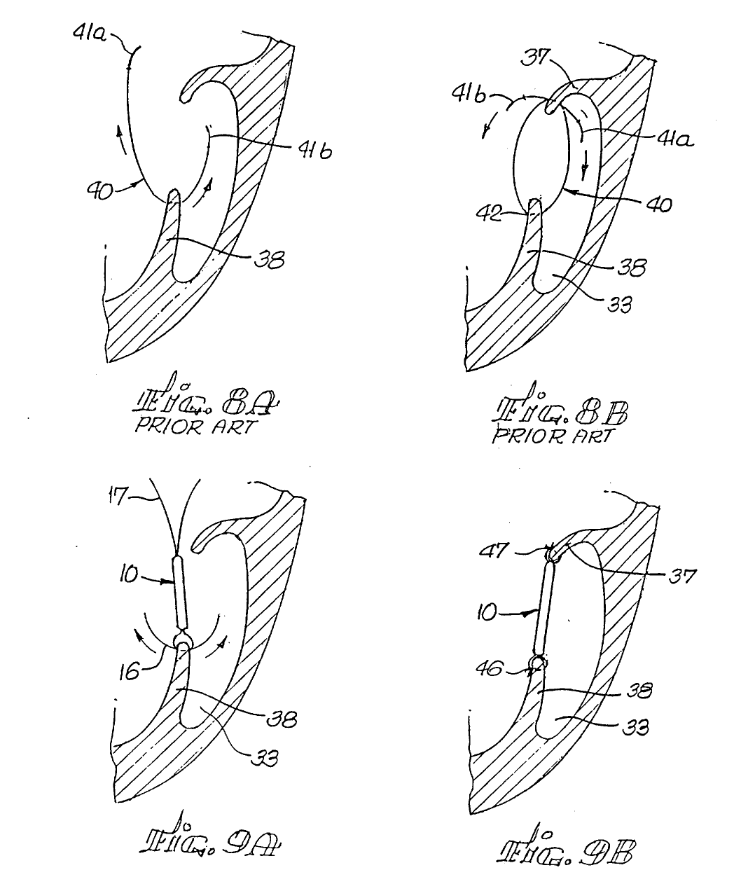

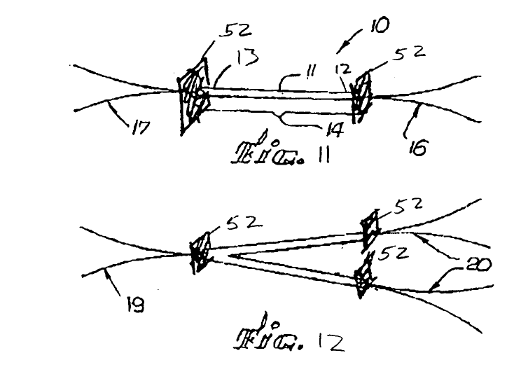

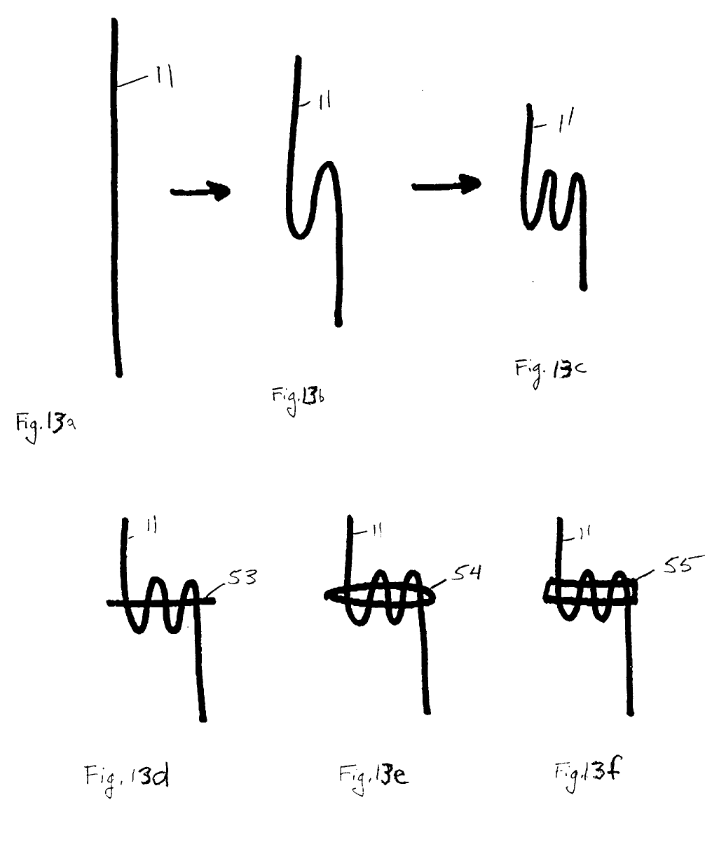

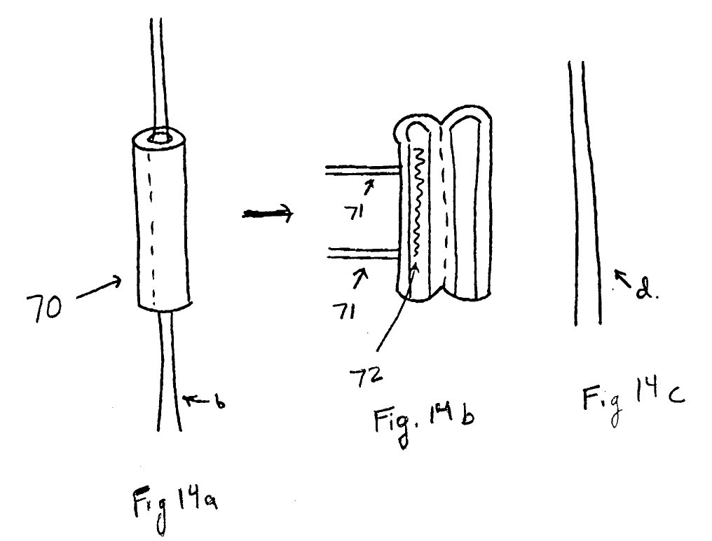

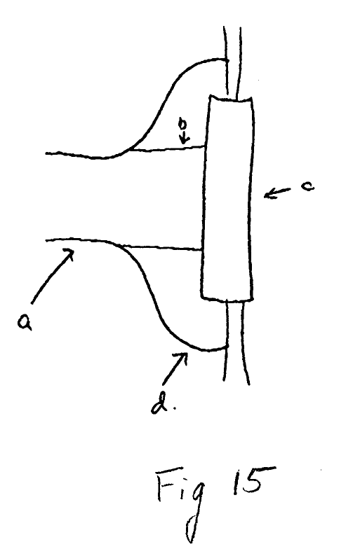

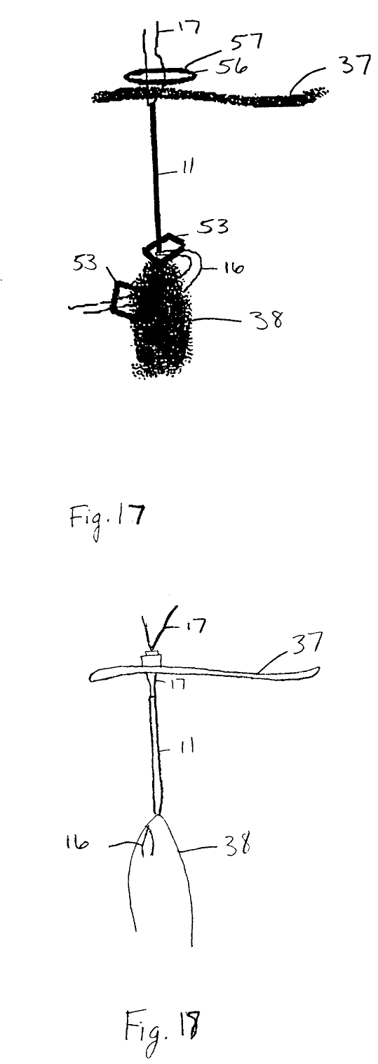

The human heart 30 is illustrated in FIG. 6, and includes the left and right atria 31, 32, and the left and right ventricle 33, 34. The mitral valve 35 is between the left atrium 31 and left ventricle 33, and the tricuspid valve 36 is similarly located between the right atrium 32 and right ventricle 34. In the mitral valve 35, the edges of the mitral valve leaflets 37 are connected to the papillary muscle 38 by the chordae tendineae 39 (FIG. 7).

FIG. 8 illustrates a sequence of steps used in attaching the prior art suture 1 in place in the heart. The suture 1 is attached in place by passing needles 2a, b through the papillary muscle 38 (FIG. 8a ) and then tied into a knot 3. The needles 2a, b are then passed through the edge of the valve leaflet 37 (FIG. 8b ), at which point a second knot is tied to secure the suture 1 in place.

FIG. 9 illustrates a series of steps used to attach the artificial chordae 10 of the invention, where the suture 16 is passed through the papillary muscle 38 secured in place with knot 46 (FIG. 9a ), and suture 17 is passed through the valve leaflet edge and secured in place with knot 47 (FIG. 9b ).

The method of replacing a chordae in a heart valve of a patient using the artificial chordae 10 of the invention comprises measuring the distance between the papillary muscle 38 and valve leaflet edge 37 using a heart valve chordae sizing gauge 21. As discussed above, the physician may measure a maximum and minimum distance between the papillary muscle 38 and valve leaflet edge 37, and calculate an average distance. An appropriately sized artificial chordae 10 is then chosen, which is surgically attached to the papillary muscle 38 and valve leaflet edge 37 at locations on the heart tissue corresponding to the location of the chordae being replaced. The first pair of sutures 16 is stitched through the papillary muscle 38 (or valve leaflet edge 37) and the sutures 16 are tied into a knot 46 so that the strand member first end 12 is secured to the papillary muscle 38 (or valve leaflet edge 37). The second pair of sutures 17 are then stitched though valve leaflet edge 37 and tied into a knot 47 to secure the strand member second end 13 to the valve leaflet edge 37.

An identical procedure is performed in the case of heart valve replacement, except that one pair of artificial chordae sutures 16,17 is attached to the valve annulus 51 of the artificial heart valve prosthesis 50 before implanting the prosthesis 50, and then the other pair of artificial chordae sutures 16,17 is attached to the original or replacement papillary muscle after the artificial heart valve prosthesis 50 is implanted. The sutures may be pledget-supported with at least one patch 52 as illustrated in FIGS. 11 and 12. The pledget may be fixedly attached to the artificial chordae strand member or sutures, or alternatively, slidably attached thereto, to facilitate positioning or suturing thereof.

In an alternative embodiment, the strand member 11 has a length that is adjustable, so that the size of the artificial chordae can be adjusted. The length may be adjusted in situ. The chordae may be fashioned as described above with one suture at each end or a plurality of sutures at each end. The chordae strand member may have a variety of configurations including tubular (cylindrical), prismatic, bifurcated, multi-subunited with multiple ends, flat sheet with single or multiple segmented end tethers and the like. The chordae strand member may be formed of a variety of materials that may be length adjusted in situ. A variety of mechanisms may be utilized for length adjustment including, but not limited to, mechanical, chemical curing, heat curing, ultrasonic curing, and the like. For mechanical length adjustment, the chordae may be made of synthetic or natural polymers or noncorrosive metal, such as flexible surgical stainless steel. The materials may be formed into tubular fibrous elements that may be either singular or woven or braided to make up the strand member. In a presently preferred embodiment, the polymers include polyethylene, polypropyine, PET, PTFE, elastin, collagen, non-immunogenic silk, spider silk, and the like. To mechanically shorten the chordae one either end, or both ends, are attached to the papillary muscle and the valve ring, the strand member will be adjusted to the clinically appropriate length arrived at by a measurement device as described, echo data, or clinical judgment. The chordae may be mechanically shortened as illustrated in FIGS. 13a-13c. The chordae may be folded over, singly or multiply, pleating or embricating the chordae. The appropriate length chordae may be then fixed at the length via a central suture, piercing pin (1b), encircling loop or ring (1c), clasplike fastener or other securing device (1d).

Further the device may be mechanically shortened by a central take-up spool like device placed over the chordae allowing shortening from either end. This device may be manually wound-up or have a central sping to apply shortening tension. This device may be composed of hemocompatabile polymeric components or stainless steel or other non-corrosive elements (1e).

To chemically shorten the chordae it is envisioned that the central member will be made of a polymeric material amenable to chemical shrinkage. Natural polymers such as polyamino acid materials, proteins, i.e. collagen, rubbers, etc. or other synthetic materials amenable to chemical shrinkage may be utilized.

One embodiment will be to expose the central member utilizing an encircling, enveloping tubular device that circulates a shrinking agent over the in situ chordae to allow shrinkage. Care would be exerted with this method to prevent leakage into the field of the curing agent. Once cured the encircling curing sleeve would rinse the chordae with physiologically appropriate solvents to allow blood and field re-exposure.

A second embodiment would place a tubular device over the chordae which provides shortening tension on both ends yet allows the central member to be exposed to a solvent. For example, a chordae is made of an aliphatic polyester that dissolves in methylene chloride or other like solvent. The central component of the central member may then be reconfigured and “shrunk” via the compaction of the encircling deice while the chordae is in a fluent state. Once at the right length the fluence of the central component may be reversed via vacuum evacuation of the solvent. Once adequate structural stability of the central member is established the encircling shrinkage device may be removed. The net result is that the chordae has been in situ remolded to a shorter but stubbier configuration.

To thermally shorten the chordae it is envisioned that the chordae may be composed of materials that eitther shrink when exposed to heat or may be remolded, i.e. similar to above though without the solvent. Heat sensitive materials include synthetic and natural polymers. To perform the in situ reconfiguration it is envisioned that an enveloping tubular member will be placed over the chordae and uniformly heated within its core. The chorde will then shrink. Materials that change from non-fluent to fluent state the device, similar to above, will have a tensioning mechanism favoring shrinkage while maintaining the central generally tubular structure of the chordae, i.e. it will act as a mold. Once reconfigured and cooled the device will be removed.

A typical chemical or thermal shrinkage device (70) for the artificial chordae is depicted in FIG. 14. The device is generally tubular to allow in situ enveloping of the chordae (1b). The device may have a single or plurality of electrical or hollow fluid conduits (71) to allow either electrical activation of a central heating element (72). Alternatively 72 may be a single or series of channels which in the closed configuration of the device (70) allows solvent or curing fluid perfusion or superfusion. Further the device may contain a central ultrasonic element, activated either peripherally or centrally to ultrasonically and/or thermally actuate the chordae. The device may be hinged (as in FIG. 14b) so that it may open and close around the chordae.

An example of an actual instrument is envisioned in FIG. 15. A surgically and ergonomically acceptable handle (1a) will be attached via a central member (1b) to the shrinkage member (1c). The shrinkage member will be central between two tethering spring-like tensioning elements (1d). These elements will tend to shorten the chordae when the central aspect of the chordae is subjected to chemical, thermal or ultrasonic energy allowing the material to creep under applied tension. While one configuration is shown it is clear that the tensioning element may be on only one end or both. The tensioning may be variable. A strain gauge or other measuring element may be incorporated to measure either the stress or the strain of the chordae so as to allow appropriate creep and reconfiguration and avoid tensile rupture of the chordae.

Thermosensitive and thermoplastic polymers may be utilized for the chordae. For example a material made of a nondegradable polymer composite with polycaprolactone would allow melting at 50-70° C. Further other thermoplastics i.e. polypropylene or polyethylene may be used and melted and recongigured in situ.

A device for changing the size of the chordae, as illustrated in FIGS. 14a-14c includes an enveloping member, a tensioning member, and a measuring device. A method of adjusting the size of the chordae comprises grasping the chordae, encircling the chordae with the tubular member, tensioning the chordae or acuating it, as by changing from nonfluent to fluent states, to reduce the size of the chordae, deactivating the chordae to make it biocompatable, and releasing the chordae, as illustrated in FIGS. 14a-14c.

Thus the length of the strand member is adjusted to correspond to the distance between the location on the papillary muscle and the location on the valve leaflet at which the ends of the strand member are attached. In one embodiment, the strand member is foldable, and the length of the strand member is adjusted by folding the strand member one or more times, as illustrated in FIGS. 13a, 13b, and 13c. FIG. 13b illustrates the strand member folded one time to decrease the length thereof, and FIG. 13c illustrates the strand member folded two times to further decrease the strand member length. The folds of the strand member are connected together to fix the strand member in the folded configuration. A variety of suitable connecting members may be used including pins, sutures, hoops or rings, clips and clamps. For example, FIG. 14 illustrates a pin 53 extending through the folds of the strand member, FIG. 15 illustrates a ring 54 positioned around the folded section of the strand member, and FIG. 16 illustrates a clip 55 positioned around the folded section of the strand member, to hold the strand member in the folded configuration. In an alternative embodiment, the length of the strand member is adjustable by heat shrinking or chemically shrinking the strand member, to decrease a length thereof. For example, the strand member can be formed of a heat shrinkable material, or the material may be chemically shrunk by solvent removal.

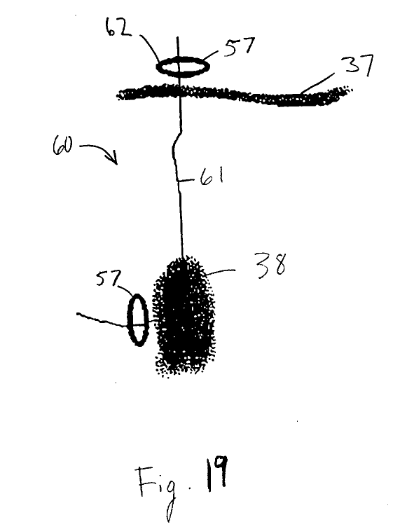

In another embodiment of the invention, illustrated in FIG. 17, an assembly is provided comprising the artificial chordae of the invention and at least one stopping member 56 configured to secure to the sutures. The stopping member is secured to the pair of sutures after the sutures are stitched through the heart tissue to prevent the sutures from slipping out of the tissue, but without the requirment of tying the two sutures into a knot. In the embodiment illustrated in FIG. 17 the stopping member comprises a clip 57 which secures to the sutures by gripping the sutures between inwardly tensioned arms of the clip. However, a variety of suitable stopping members may be used including clamps, rings, hoops, and the like. For example, FIG. 18 illustrates an alternative embodiment in which the stopping member comprises a tube 58 having a bore configured to slidably receive one or more of the sutures of the pair of sutures, and having a fastening member, such as a fastener having a variable inner diameter with a reduced inner diameter configuration which frictionally engages the suture, to secure the suture to the tube.

In the embodiment illustrated in FIG. 18 the stopping member is secured to the second pair of sutures 17 along a length thereof so that a length of the sutures 17 extends between the heart valve leaflet edge and the papillary muscle. The stopping member is configured to quickly and easily secure to the sutures, so that the stopping member can be used to hold the suture in place without the length of the suture spanning the distance between the papillary muscle and valve leaflet changing. Thus, even if the length of the strand member is not correctly sized to correspond to the distance between the papillary muscle and the valve leaflet edge, the artificial chordae can be implanted using the stopping member so that a combined length of the strand member and the sutures is correctly sized to correspond to the distance between the muscle and valve leaflet. For example, the physician can attach the first end of the strand member to the papillary muscle, stitch the second pair of sutures through the valve leaflet so that the strand member or the strand member and a length of the second pair of sutures corresponds to the distance between the papillary muscle and the attachment location on the valve leaflet, and secure the stopping member to the second pair of sutures quickly and without longitudinally displacing the second pair of sutures further one way or another through the valve leaflet. It would be obvious to one of ordinary skill in the art that one or more stopping members may be used on one or both of the first 16 and second 17 pair of sutures.

Thus, the artificial chordae of the invention may be provided in two or three different sizes having strand members with different lengths, so that the physician can choose an artificial chordae that is approximately the correct size and then adjust the size, as described above, to more exactly fit the patient.

In an alternative embodiment of the invention, illustrated in FIG. 19, the artificial chordae 60 comprises a suture 61 having a first end and a second end, and at least one stopping member 62 on either end thereof configured to secure to the suture. As discussed above, the stopping member can be secured to the suture to hold it in place without the disturbing or changing the length of the suture spanning the distance between the papillary muscle and valve leaflet. In the method of attaching the artificial chordae 60, the suture 61, which may be formed using conventional suture materials and dimensions, first end is stitched through the papillary muscle from a first side to a second side of the muscle, and the first stopping member is positioned on the first end of the suture adjacent to second side of the muscle, and the stopping member is secured to the suture. The second end of the suture is similarly stitched through the valve leaflet edge so that a length of the suture conforms to the length between the papillary muscle and valve leaflet edge. The second stopping member is then secured to the second end of the suture as above, without longitudinally displacing the suture and changing the length of the suture between the papillary muscle and the valve leaflet edge. In the embodiment illustrated in FIG. 19, the stopping member comprises a clip 57, as discussed above. Thus, the artificial chordae can be correctly sized and implanted quickly and easily.

While the present invention has been described in terms of certain preferred embodiments, those skilled in the art will recognize that modifications and improvements may be made to the invention without departing from the scope thereof. For example, the artificial chordae may be made of a plurality of braided strands, a biopolymer or a biopolymer-synthetic composite, including degradable or nondegradable materials which may be physical blends or copolymers.

What is claimed is:

1. Artificial chordae for a heart valve, comprising:
a) at least one strand member having a first end and a second end, and being configured to extend from a papillary muscle to a location on the heart valve; and
b) a first pair of sutures extending from the first end of the strand member and a second pair of sutures extending from the second end of the strand member.

2. The artificial chordae of claim 1 wherein the location on the heart valve is a valve leaflet edge.

3. The artificial chordae of claim 1 wherein the strand member is from about 75 cm to about 90 cm in length.

4. The artificial chordae of claim 1 wherein the sutures are from about 1 cm to about 6 cm in length.

5. The artificial chordae of claim 1 wherein the strand member and the sutures are formed from one unitary piece of material.

6. The artificial chordae of claim 1 wherein the strand member and the sutures are formed from expanded polytetrafluoroethylene.

7. The artificial chordae of claim 6 wherein the expanded polytetrafluoroethylene is selected from the group consisting of polytetrafluoroethylene suture material and polytetrafluoroethylene fabric.

8. The artificial chordae of claim 1 having at least two strand members, with the first ends of the strand members fixed together to form a joined end, wherein the strand members are longitudinally juxtaposed, and having one pair of sutures extending from the joined end, and a pair of sutures extending from the second end of each strand member.

9. The artificial chordae of claim 8 wherein the strand members are of equal lengths.

10. The artificial chordae of claim 1 wherein at least one pair of sutures includes a pledget at an interface between the sutures and the stand member.

11. The artificial chordae of claim 1 wherein at least one pair of sutures includes a stopping member configured to secure to the sutures.

12. The artificial chordae of claim 11 wherein the stopping member comprises a clip configured to grippingly secure to the pair of sutures.

13. The artificial chordae of claim 1 wherein the stopping member comprises a tube having a bore configured to slidably receive one or more of the sutures of the pair of sutures, and having a fastening member to secure the suture to the tube.

14. The artificial chordae of claim 1 wherein the strand member has a length that is adjustable.

15. The artificial chordae of claim 15 wherein the strand member is formed of a material that is heat shrinkable or chemically shrinkable.

16. The artificial chordae of claim 15 wherein the strand member is foldable and including a connecting member for connecting one or more folds of the strand member together.

17. The artificial chordae of claim 16 wherein the connecting member is selected from the group consisting of pins, sutures, and clamps, rings.

18. A heart valve chordae sizing gauge for measuring the distance between a papillary muscle and a location on a heart valve, comprising a shaft having a first end and a second end, and a transverse member spaced a distance between the first and second ends of the shaft.

19. The sizing gauge of claim 10 wherein the distance between the transverse member and the second end of the shaft is substantially equal to the distance between the papillary muscle and a valve leaflet edge of the heart valve.

20. The sizing gauge of claim 10 wherein the transverse member is mounted so as to slide along the shaft, and further including a means for releasably locking the transverse member onto the rod.

21. The sizing gauge of claim 10 having a handle on the first end of the shaft.

22. A method of attaching an artificial chordae in a heart, comprising:
a) providing an artificial chordae, comprising:
at least one strand member having a first end and a second end, and configured to extend from a papillary muscle to a location on the heart valve; and
a first pair of sutures extending from the first end of the strand member and a second pair of sutures extending from the second end of the strand member; and
b) attaching the sutures to the papillary muscle and to the heart valve, to attach the artificial chordae in the heart.

23. The method of claim 22 wherein the step of attaching the sutures further comprises:
a) stitching the first pair of sutures through a valve leaflet edge and tying the two sutures into a knot so that the first end of the strand member is secured to the valve leaflet edge; and
b) stitching the second pair of sutures through the papillary muscle and tying the two sutures into a knot so that the second end of the strand member is secured to the papillary muscle.

24. The method of claim 23 wherein the artificial chordae is attached by first attaching the first pair of sutures to a valve annulus of a heart valve prosthesis before the heart valve prosthesis is implanted, and then attaching the second pair of sutures to the papillary muscle after the heart valve prosthesis is implanted.

25. The method of claim 22 including, before step a, the step of measuring the distance between the papillary muscle and the location on the heart valve with a heart valve chordae sizing gauge, the gauge comprising a shaft having a first end and a second end, and a transverse member spaced a distance between the first and second ends of the shaft.

26. The method of claim 25 wherein the measuring step comprises holding the sizing gauge between the papillary muscle and a valve leaflet edge so that the second end of the sizing gauge contacts the papillary muscle and sliding the transverse member along the shaft until the member contacts the valve leaflet edge.

27. The method of claim 22 wherein at least one pair of sutures includes a stopping member configured to secure to the sutures, and wherein the step of attaching the sutures to the papillary muscle includes the step of stitching the pair of sutures through the papillary muscle from a first side to a second side of the papillary muscle, and securing the stopping member to the suture at a location on the suture adjacent the second side of the papillary muscle, to thereby prevent the displacement of the suture from the second side to the first side of the papillary muscle.

28. The method of claim 22 wherein at least one pair of sutures includes a stopping member configured to secure to the sutures, and wherein the step of attaching the sutures to the heart valve includes the step of stitching the pair of sutures through a valve leaflet edge from a first side to a second side of the valve leaflet edge, and securing the stopping member to the suture at a location on the suture adjacent the second side of the valve leaflet edge, to thereby prevent the displacement of the suture from the second side to the first side of the valve leaflet edge.

29. The method of claim 22 wherein the strand member has a length that is adjustable, and including the step of adjusting the length of the strand member to conform to a length between the papillary muscle and a location of the heart valve.

30. The method of claim 29 wherein the step of adjusting the length of the strand member includes the step of folding a length of the strand member, and connecting the folds together to decrease the length of the strand member.

31. The method of claim 29 wherein the step of adjusting the length of the strand member includes heat shrinking or chemically shrinking the strand member to decrease the length of the strand member.

32. An artificial chordae for a heart valve of a patient's heart, comprising:
a) a suture having a first end and a second end; and
b) a first stopping member on the first end, and a second stopping member on the second end, each securing member being configured to secure to the suture, to thereby secure the suture within the patient's heart.

33. The artificial chordae of claim 32 wherein the stopping member comprises a clip configured to grippingly secure to the suture.

34. The artificial chordae of claim 32 wherein the stopping member comprises a tube having a bore configured to slidably receive the suture, and having a fastening member to secure the suture to the tube.

35. A method of attaching an artificial chordae in a patient's heart, comprising:
a) providing an artificial chordae comprising
a suture having a first end and a second end; and
a first stopping member on the first end and a second stopping member on the second end, each stopping member being configured to secure to the suture for securing the suture within the patient's heart;
b) attaching the first end of the suture to a papillary muscle of the patient's heart by stitching the first end of the suture through the papillary muscle from a first side of the muscle to a second side of the muscle, and positioning the stopping member at a location on the suture adjacent the second side of the papillary muscle, and securing the stopping member to the suture to thereby prevent the displacement of the suture from the second side to the first side of the papillary muscle; and
d) attaching the second end of the suture to a valve leaflet edge of the patient's heart by stitching the second end of the suture through the valve leaflet edge at a location on the valve leaflet edge from a first side of the valve leaflet edge to a second side of the valve leaflet edge so that a length of suture conforms to a length between the papillary muscle and the location on the valve leaflet edge, and positioning the stopping member at a location on the suture adjacent the second side of the valve leaflet edge, and securing the stopping member to the suture to thereby prevent the displacement of the suture from the second side to the first side of the valve leaflet edge.
